(19) United States
(12) Patent Application Publication
(10) Pub. No.: US 2008/0188549 A1
(43) Pub. Date: Aug. 7, 2008

(54) Composition for Preventing and/or Treating Metabolic Syndrome and Insulin Resistance Syndrome

(75) Inventors: Hozumi Tanaka, Hyogo (JP); Shinichi Yokota, Hyogo (JP); Misuzu Tsukagawa, Hyogo (JP); Tatsumasa Mae, Hyogo (JP); Minpei Kuroda, Tokyo (JP); Yoshihiro Mimaki, Tokyo (JP)

Correspondence Address: CONNOLLY BOVE LODGE & HUTZ LLP, 1875 EYE STREET, N.W., SUITE 1100, WASHINGTON, DC, 20036, US

(73) Assignee: Kaneka Corporation, Kita-ku, Osaka-shi (JP)

(21) Appl. No.: 11/815,896

(22) PCT Filed: Feb. 8, 2006
(86) PCT No.: PCT/JP2006/302190

§ 371 Date: Mar. 19, 2008

(30) Foreign Application Priority Data
Feb. 9, 2005 (JP) 2005-032737

(51) Int. Cl. A61K 31/352; C07C 49/248; C07D 311/26; A61P 3/00; A61K 31/12; C07D 493/04

(52) U.S. Cl. 514/455; 568/325; 549/387; 549/403; 514/456; 514/685

(57) ABSTRACT

The present invention has its object to provide a specific compound derived from a naturally occurring substance, and a composition for the prevention and/or treatment of metabolic syndrome and insulin resistance syndrome which comprises that as an active ingredient, easily and efficiently.

The present invention relates to a composition for the prevention and/or treatment of metabolic syndrome and a composition for the prevention and/or treatment of insulin resistance syndrome,
each of which comprises, as an active ingredient, at least one compound selected from among the compound represented by the formula (1), compounds represented by the formula (4) and compounds represented by the formula (5), or a salt or ester thereof.

### TECHNICAL FIELD

The present invention relates to a novel compound, a composition for the prevention and/or treatment of metabolic syndrome, and a composition for the prevention and/or treatment of insulin resistance syndrome.

### BACKGROUND ART

Peroxisome proliferator-activated receptor (PPAR) is a ligand-dependent transcriptional regulatory factor belonging to a family of nuclear receptors identified as transcriptional regulatory factors controlling the expression of a group of genes for maintaining lipid metabolism. In mammalian animals, the presence of 3 subtypes, i.e. PPARα, PPARδ (PPARβ, NUC-1, FAAR) and PPARγ, is known, and PPARα is expressed mainly in the liver and PPARδ is universally expressed. For PPARγ, there are 2 isoforms, i.e. PPARγ1 and PPARγ2, and PPARγ1 is expressed not only in an adipose tissue but also in an immune organ, adrenal gland and small intestine. PPARγ2 is expressed specifically in an adipose tissue, and is a master regulator which regulates differentiation and maturation of an adipocyte (cf. Non-Patent Document 1).

As PPARγ ligands, arachidonic acid metabolites such as 15-deoxy-Δ12,14-prostaglandin J2 and Δ12-prostaglandin J2, unsaturated fatty acids such as ω-3 multivalent unsaturated fatty acid, α-linolenic acid, eicosapentaenoic acid (EPA) and docosahexaenoic acid (DHA), and eicosanoids such as 9-hydroxyoctadecadienoic acid and 13-hydroxyoctadecadienoic acid are known (cf. Non-Patent Document 2). In addition, a C₁₀-26 conjugated unsaturated fatty acid having a conjugated triene structure or a conjugated tetraene structure is described (cf. Patent Document 1). Synthetic compounds including thiazolidine derivatives such as troglitazone, pioglitazone and rosiglitazone are known to be PPARγ ligands.

The agonistic activity of a thiazolidine derivative as PPARγ ligand is correlated with its hypoglycemic action, thus the derivative has attracted attention for its relationship with a treating action on insulin resistance syndrome and been developed as an insulin resistance syndrome treating agent for type 2 diabetes mellitus (non-insulin dependent diabetes mellitus: NIDDM). That is, a thiazolidine derivative, which is a PPARγ ligand, activates PPARγ thereby increasing a normally functioning small adipocyte differentiated from a preadipocyte. Thereafter, a hypertrophic adipocyte, in which over-production and over-secretion of TNFα and a free fatty acid inducing insulin resistance syndrome, is reduced with apotosis. As a result, insulin resistance syndrome is treated (cf. Non-Patent Document 3). A PPARγ ligand is thus effective in preventing and/or treating the insulin resistance syndrome including not only type 2 diabetes mellitus but also hyperinsulinemia, dyslipidemia, obesity, hypertension and arteriosclerosis (cf. Non-Patent Document 4). A PPARγ ligand is also effective in preventing and/or treating inflammations and cancers because of its inhibition of inflammatory cytokine production (Non-Patent Document 5) and its induction of apotosis thereby inhibiting growth of cancer cells (cf. Non-Patent Document 6). Further, in recent years, chemically synthesized PPARγ modulators which can be expected to show pharmacological actions different from those of thiazolidine derivatives like troglitazone have been under development (cf. Non-Patent Document 7).

A PPARγ ligand has an effect of preventing and/or treating the metabolic syndrome and insulin resistance syndrome including not only type 2 diabetes mellitus but also hyperinsulinemia, dyslipidemia, obesity, hypertension and arteriosclerosis.

Patent Document 1: Japanese Kokai Publication 2000-355538

Non-Patent Document 1: Teruo Kawada, “Igaku No Ayumi”, 184, 519-523, 1998

Non-Patent Document 2: J. Auwerx, Diabetologia, 42, 1033-1049, 1999

Non-Patent Document 3: A Okuno, et al., Journal of Clinical Investigation, 101, 1354-1361, 1998

Non-Patent Document 4: R. A. Degronzo et al., Diabetes Care, 14, 173-194, 1991

Non-Patent Document 5: C. Jiang, et al., Nature, 391, 82-86, 1998

Non-Patent Document 6: Y. Tsubouchi, et al., Biochemical and Biophysical Research Communications, 270, 400-405, 2000

Non-Patent Document 7: Molecular Endocrinology, 17, 662-676, 2003

### SUMMARY OF THE INVENTION

In view of the foregoing, it is an object of the present invention to provide a specific edible compound derived from a naturally occurring substance; a PPARγ ligand agent comprising the specific compound as an active ingredient; and a composition for the prevention and/or treatment of metabolic syndrome and insulin resistance syndrome which comprises the specific compound as an active ingredient.

The present inventors found that licorice extract and certain like plant extracts have hypoglycemic and lipid metabolism treatment effects and that such extracts have PPARγ ligand activity. The present inventors earnestly searched for active ingredients contained therein and, as a result, found that certain specific ingredients in the extracts have PPARγ ligand activity and, further, that in extracting these ingredients from licorice, licorice can be efficiently extracted with an amphiphilic solvent. These findings have led to completion of the present invention.

Thus, the present invention relates to

a compound represented by the formula (1) or a salt or ester thereof:

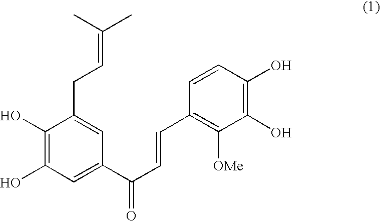

a compound represented by the formula (2) or a salt or ester thereof:

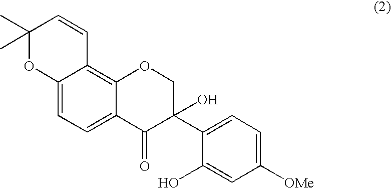

and a PPARγ ligand agent

which comprises, as an active ingredient, at least one compound selected from among the compound represented by the formula (1), compounds represented by the formula (4) and compounds represented by the formula (5), or a salt or ester thereof:

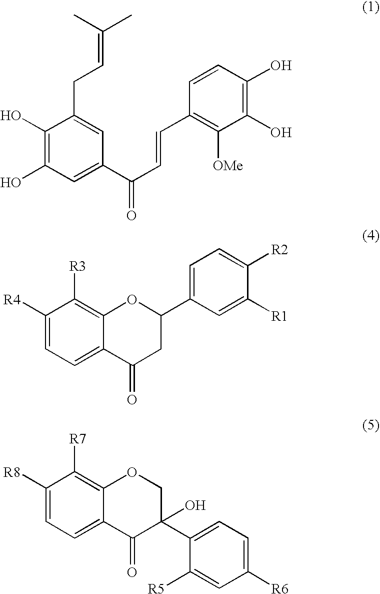

[In the above formulae, at least one of R1 to R4 is a prenyl group or at least one pair of neighboring two groups together represent —CH═CHC(CH₃)₂O— to form a six-membered ring and the others each independently represents H, OH, OCH₃ or CHO. R5 and R6 each independently represents H, OH or OCH₃. At least one of R7 and R8 is a prenyl group or R7 and R8 together represent —CH═CHC(CH₃)₂O— to form a six-membered ring; the other represents H, OH, OCH₃ or CHO.]

The invention also relates to

a composition for the prevention and/or treatment of metabolic syndrome and a composition for the prevention and/or treatment of insulin resistance syndrome,

each of which comprises, as an active ingredient, at least one compound selected from among the compound represented by the formula (1), compounds represented by the formula (4) and compounds represented by the formula (5), or a salt or ester thereof;

a composition for the prevention and/or treatment of metabolic syndrome and a composition for the prevention and/or treatment of insulin resistance syndrome, each as defined above,

wherein the compound represented by the formula (4) is a compound represented by the formula (6):

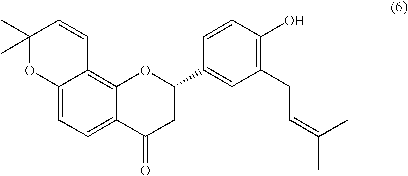

a composition for the prevention and/or treatment of metabolic syndrome and a composition for the prevention and/or treatment of insulin resistance syndrome, each as defined above,

wherein the compound represented by the formula (5) is the compound represented by the formula (2): and

a composition for the prevention and/or treatment of metabolic syndrome and a composition for the prevention and/or treatment of insulin resistance syndrome, each of which is as defined above and

which comprises at least one compound selected from among the compound represented by the formula (1), compounds represented by the formula (4) and compounds represented by the formula (5), or a salt or ester thereof, the content thereof being not lower than 0.0000001% by weight but lower than 100% by weight.

The invention further relates to:

a composition as defined above

which is to be eaten or drunk;

a composition as defined above,

which is used for medical or medicinal purposes;

a composition as defined above,

which is applied to domestic animals or pets;

a method for the prevention and/or treatment of metabolic syndrome and a method for the prevention and/or treatment of insulin resistance syndrome,

each of which comprises using at least one compound selected from among the compound represented by the formula (1), compounds represented by the formula (4) and compounds represented by the formula (5), or a salt or ester thereof; and

the use of at least one compound selected from among the compound represented by the formula (1), compounds represented by the formula (4) and compounds represented by the formula (5), or a salt or ester thereof for the preparation of a composition for the prevention and/or treatment of metabolic syndrome and of a composition for the prevention and/or treatment of insulin resistance syndrome.

### DETAILED DESCRIPTION OF THE INVENTION

In the following, the present invention will be described in detail.

Each of the composition for the prevention and/or treatment of metabolic syndrome and a composition for the prevention and/or treatment of insulin resistance syndrome according to the present invention comprises, as an active ingredient, at least one compound selected from among the compound represented by the formula (1), compounds represented by the formula (4) and compounds represented by the formula (5), or a salt or ester thereof:

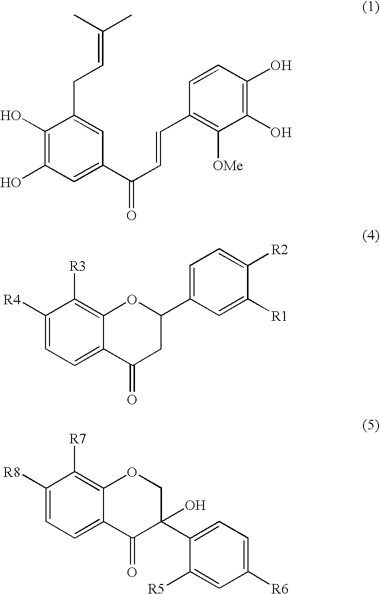

[In the above formulae, at least one of R1 to R4 is a prenyl group or at least one pair of neighboring two groups together represent —CH═CHC(CH₃)₂O— to form a six-membered ring and the others each independently represents H, OH, OCH₃ or CHO. R5 and R6 each independently represents H, OH or OCH₃. At least one of R7 and R8 is a prenyl group or R7 and R8 together represent —CH═CHC(CH₃)₂O— to form a six-membered ring; the other represents H, OH, OCH₃ or CHO.]

The compound represented by the formula (1), the compounds represented by the formula (4), the compounds represented by the formula (5), and salts and esters thereof have PPARγ ligand activity and can serve as PPARγ ligand agents to be used in the present invention.

The term “treatment” as used herein means cure or alleviation.

The peroxisome proliferator-activated receptor (PPAR) so referred to herein is a ligand-dependent transcriptional regulatory factor belonging to a family of nuclear receptors identified as transcriptional regulatory factors controlling the expression of a group of genes for maintaining lipid metabolism, as mentioned hereinabove. PPARγ, one of the subtypes thereof, includes two isoforms, namely PPARγ1 and PPARγ2, in mammals, and PPARγ1 is expressed not only in adipose tissues but also in immune system organs, adrenal gland and small intestine.

The term “PPARγ ligand agent” as used herein means a compound having affinity for PPARγ (compound having PPARγ ligand activity).

Whether a compound has PPARγ ligand activity or not can be determined by the assay described in Example 2 to be given later herein.

The term “metabolic syndrome” as used herein means the syndrome involving, in addition to visceral fat obesity, at least one concurrent morbid state selected from the group consisting of diabetes, hyperlipemia and hypertension. The term is synonymous with “visceral fat syndrome” and “multiple risk factor syndrome” (cf. Matsuzawa et al., Diabetes/Metabolism Reviews, 13, 3-13, 1997).

The term “insulin resistance syndrome” as used herein means the syndrome involving at least two concurrent morbid states selected from the group consisting of hyperinsulinemia, lipid metabolism abnormalities, obesity, hypertension and arteriosclerotic diseases (cf. R. A. Degronze et al., Diabetes Care, 14, 173-194, 1991).

The compound represented by the formula (1) to be used in the present invention is a novel compound:

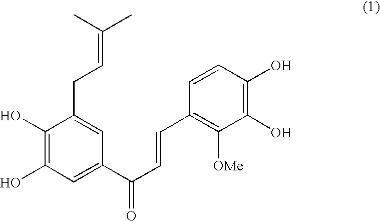

The compound represented by the formula (4) to be used in the present invention is a flavanone derivative:

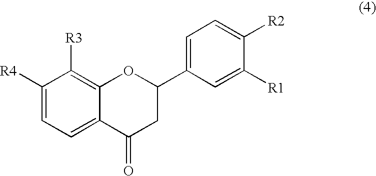

[In the above formula, at least one of R1 to R4 is a prenyl group or at least one pair of neighboring two groups together represent —CH═CHC(CH₃)₂O— to form a six-membered ring and the others each independently represents H, OH, OCH₃ or CHO.]: and among them, shinflavanone represented by the formula (6) is particularly preferred:

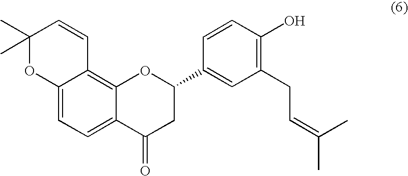

The compound represented by the formula (5) to be used in the present invention is an isoflavanone derivative:

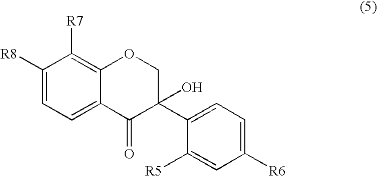

[In the above formula, R5 and R6 each independently represents H, OH or OCH₃. At least one of R7 and R8 is a prenyl group or R7 and R8 together represent —CH═CHC(CH₃)₂O— to form a six-membered ring; the other represents H, OH, OCH₃ or CHO.]: and among them, the novel compound represented by the formula (2) is particularly preferred:

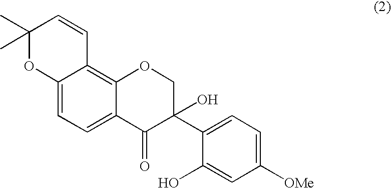

Salts of any of the above-mentioned compounds, if the compound is basic, can also be suitably used in the practice of the invention. The salts can be formed by mixing a solution of each of the above-mentioned compounds with a solution of an appropriate acid acceptable for drinking or eating, for medical use, or in preparing feeds or pet foods (e.g. hydrochloric acid, sulfuric acid, methanesulfonic acid, fumaric acid, maleic acid, succinic acid, acetic acid, benzoic acid, oxalic acid, citric acid, tartaric acid, carbonic acid, phosphoric acid). When the compound has an acidic moiety, the salts thereof acceptable for drinking or eating, for medical use, or in preparing feeds or pet foods include alkali metal salts (e.g. sodium salt, potassium salt); alkaline earth metal salts (e.g. calcium salt, magnesium salt); and salts formed with appropriate organic ligands (e.g. quaternary ammonium salts).

Esters, such as fatty acid esters, of the above-mentioned compounds can also be properly used in the practice of the invention. The esters can be formed with arbitrary organic acids or inorganic acids. Usable as the acids are acids suitable for drinking or eating, for medical use, or in preparing feeds or pet foods; preferred are fatty acids. The fatty acids include, but are not limited to, long-chain fatty acids such as oleic acid, palmitic acid, stearic acid, linolic acid and linolenic acid; short- or medium-chain fatty acids such as acetic acid and butyric acid.

The above-mentioned compounds may be plant- or some other naturally occurring substance-derived compounds or chemically synthesized compounds, preferably naturally occurring substance-derived edible compounds.

The method of producing the above-mentioned compounds is now described.

The method of producing the above-mentioned compounds is not particularly restricted. For example, the above-mentioned compounds can be obtained by extracting from a plant which is a kind of licorice and belongs to the family Leguminosae, genus Glycyrrhiza. The compounds may also be extracted from some other plant or may be chemically synthesized.

In the case of obtaining the compound from licorice, the usable licorice may be a plant of the genus Glycyrrhiza in the Leguminosae family, and examples thereof include Glycyrrhiza uralensis Fisch. et DC, G. inflata BAT., G. glabra L., G. glabra L. var glandu rifera Regel et Herder, G. echinata L., G. pallidiflora Maxim, and other plants of the same genus (Leguminosae). Preferred are Glycyrrhiza uralensis Fisch. et DC, G. inflata BAT., and G. glabra L. etc. The licorice grown in Xin Jiang, Northeast China, Northwest China, Mongol, Russia, Afghanistan, etc. may be used, but there is no limitation.

In the practice of the invention, the root, rhizome or stolons of such a licorice species as mentioned above or, in certain cases, the plant deprived of the periderm (peeled licorice), among others, is preferably used. These are used in the form of small pieces or chips or, preferably, powders; generally, they are used in the form of small pieces or chips not larger than about 10 mm, or powders with an average particle diameter of not greater than 1,000 microns (preferably not greater than 500 microns, more preferably not greater than 200 microns). The composition and content of the active ingredient of the licorice vary to some degree depending on the species, growing area, harvest time, etc. thereof. Therefore, it is preferable to use one confirmed to contain a large amount of the active ingredient by a preliminary experiment.

As the extraction methods, there may be mentioned, but not limited to, methods which comprise extracting the licorice with an organic solvent or water, and the like methods.

As for the organic solvent to be used for the above extraction, amphiphilic organic solvents are preferred from the viewpoint of efficient extraction of the compounds mentioned above. The amphiphilic organic solvent is an organic solvent miscible with both a hydrophilic solvent and a hydrophobic solvent. As the amphiphilic organic solvents, there may be mentioned, for example, ketones (e.g. acetone), alcohols (mono-, di-, tri and polyhydric alcohols containing 1 to 4 carbon atoms, for example methanol, ethanol, propanol, butanol, propylene glycol, glycerol), and esters (e.g. ethyl acetate). These solvents may be used singly or two or more of them may be used in admixture.

Preferred as the extracting solvent are solvents comprising one or two or more species selected from among water, alcohols and acetone. Ethanol is particularly preferred since it is suited for medical use or drinking or eating.

Although the amphiphilic organic solvent may be used in an anhydrous condition, the amphiphilic organic solvent is preferably used in a hydrous condition, namely as a mixture with water. In this case, the weight ratio (amphiphilic organic solvent)/(amphiphilic organic solvent+water) is generally not lower than 0.3, preferably not lower than 0.4, more preferably not lower than 0.5, still more preferably not lower than 0.6 in consideration of extraction easiness. This ratio can be understood as the ratio in the system on the occasion of extraction. By using a hydrous solvent at a ratio within the above range, it becomes possible to reduce the amphiphilic organic solvent-due cost in the extraction step.

It goes without saying that some other solvent and/or a soluble component may be allowed to coexist so long as no adverse effects are produced thereby.

As the other solvent, there may be mentioned, for example, hexane and the like.

As the soluble component, there may be mentioned, for example, Tween species and the like.

Extraction with the amphiphilic organic solvent mentioned above can be carried out according to a general method, and is not particularly limited. The extraction procedure may be carried out, for example, for about 0.1 hour to 1 week or more by using the amphiphilic organic solvent in 1- to 20-fold excess based on the volume of the licorice. Extraction may be carried out once or several times if necessary, and a suitably combined mixed amphiphilic solvent may be used. The extraction temperature is not particularly limited, and extraction can be carried out preferably between the solidification temperature and the boiling point in the system, generally −20 to 100° C., usually 1 to 80° C. The pressure at the time of extraction is not particularly limited, and extraction is carried out at normal pressures or under pressure (1 to 5 atmospheric pressure), but may be carried out under reduced pressure if desired. The extraction may be preferably carried out under reflux or in a slightly pressurized state. The extraction procedure may be preferably carried out at pH ranged from acid to weak alkaline, preferably from acid to neutral, from the safety viewpoint.

After the extraction, the preliminary extract can be separated into an extract and an extracted licorice residue by a general separation procedure according to need (e.g. filtration under pressure, vacuum filtration, centrifugation, sedimentation, etc.) and if necessary washed with the solvent, to give a licorice extract. In the separation procedure, generally usable filter aids and the like such as activated carbon and activated clay can be used if necessary.

From the licorice extract thus obtained, the used solvent can be removed by a general procedure for solvent removal if necessary (e.g. concentration at normal pressure, vacuum concentration, spray drying, freeze drying, freeze concentration, etc.), whereby a solvent-free licorice extract can be obtained. In the process mentioned above, from the viewpoint of the active ingredient safety, a series of the above procedures, particularly extraction with an amphiphilic organic solvent etc., or extraction with an amphiphilic organic solvent etc. and subsequent procedures (separation of an extract, removal of the solvent), are preferably carried out under a deoxidized atmosphere such as an inert gas atmosphere using a nitrogen gas, etc.

Further, the desired product(s) can be fractionated or isolated and purified from the extract obtained in the above manner by silica gel, ODS or like column chromatography. More specifically, the extract fraction is concentrated under reduced pressure to remove the solvent and give a crude fraction. The desired product(s) can be isolated and purified from the crude fraction by repeating silica gel column chromatography, ODS silica gel column chromatography or high-performance liquid chromatography using an ODS column, or gel filtration column chromatography, for instance.

In a preferred mode of embodiment, the dried raw plant of Glycyrrhiza glabra, for instance, is used and extracted with an amphiphilic organic solvent (e.g. ethanol), and the extract is concentrated under reduced pressure to remove the solvent and give a dried extract. The extract is subjected to silica gel column chromatography, followed by elution with an organic solvent to give a desired compound-containing fraction. A specific example of the method of producing the compounds mentioned above is described in Example 1 given later herein.

The eluent to be used in various chromatography techniques is not particularly restricted but includes those mentioned above as the extracting solvents (water, amphiphilic organic solvents), chloroform, acetonitrile and hexane, among others.

The above-mentioned compounds can also be obtained by utilizing appropriate methods of chemical synthesis.

The above-mentioned compounds obtained by extraction from the plant mentioned above or by chemical synthesis can be converted to salts and esters thereof. As mentioned hereinabove, the salts can be obtained by admixing a solution of an appropriate acid acceptable for drinking or eating, for medical use, or in preparing feeds or pet foods with a solution of any of the above-mentioned compounds. The esters can be obtained in the conventional manner using an acid appropriate for drinking or eating, for medical use, or in preparing feeds or pet foods, as mentioned above.

The method of preparing a composition for the prevention and/or treatment of metabolic syndrome and insulin resistance syndrome according to the invention is now described. The method of preparing such composition comprises producing any of the compounds mentioned above or a salt or ester thereof and blending that compound, salt or ester with a carrier according to need.

The content of the compound, salt or ester in the composition is not particularly restricted but preferably is not lower than 0.0000001% by weight but lower than 100% by weight.

The above compound represented by the formula (1), (2), (4), (5), (6) may be a pure compound or a semi-purified or crude compound provided that it does not contain impurities unsuitable for pharmaceutical preparations and foods or beverages. In this case, the licorice extract may be used as it is, or may be further purified.

The compound is not particularly limited in its form and can be used as, for example, foods or beverages such as food with health claims (food for specified health uses, food with nutrient functional claims), health foods and dietary supplements, pharmaceutical preparations, and quasi drugs.

The compound of the present invention, and the salt, ester and composition thereof can be administered or applied to fish, reptiles, amphibians, feathers, mammals and all animals. The mammals are not particularly restricted and there may be mentioned, for example, humans, monkeys, dogs, cats, bovine species, equine species, swine species, sheep, mice, rats, and guinea pigs.

For use as a food or a beverage, it can be directly ingested or may be formulated into easily ingestable products, such as capsules, tablets, granules, etc., with the aid of a known carrier, an auxiliary agent or the like for ingestion. Furthermore, other material for preparation, which may be used for foods or beverages, can be suitably added and mixed by a conventional method. Such material is not particularly restricted, and there may be mentioned, for example, excipients, disintegrators, lubricants, binders, antioxidants, coloring agents, aggregation inhibitors, absorption promoters, solubilizing agents, stabilizers, etc.

The amount of compound of the present invention, or the salt or ester thereof in such a formulated product may be preferably not lower than 0.1% by weight but lower than 100% by weight, more preferably 10 to 90% by weight.

Furthermore, it can be mixed into raw materials for all kinds of food or beverage products, for example, confections such as chewing gum, chocolate, candies, jellies, biscuits and crackers; frozen sweets such as ice cream and ice candies; beverages such as tea, nonalcoholic beverages, nutritional drinks, and drinks for beauty; noodles such as Japanese wheat noodles, Chinese noodles, spaghetti, and instant noodles; fish paste foods such as fish minced and steamed (kamaboko), fish sausage (chikuwa), and minced flesh (hannpen); seasonings such as dressings, mayonnaise and sauces; oleaginous products such as margarine, butter and saladoil; bakery products, hams, soups, retort foods, frozen foods, and so forth.

In taking such a food or beverage composition, the recommended daily intake for an adult human is not particularly limited but is preferably 0.1 to 3,000 mg/kg, more preferably 1 to 300 mg/kg, as the compound of the invention, or the salt or ester thereof. Such compositions can also be used as feeds for domestic animals and pets or as pet foods, and the recommended daily intake in these applications is not particularly limited but is preferably 0.1 to 3,000 mg/kg as the compound of the invention, or the salt or ester thereof.

For use as a pharmaceutical product, the dosage form is not particularly restricted but includes capsules, tablets, granules, injections, suppositories, patches, etc. Such dosage forms can be prepared by suitably formulating pharmaceutically acceptable material for preparation such as excipients, disintegrators, lubricants, binders, antioxidants, coloring agents, aggregation inhibitors, absorption promoters, solubilizing agents and stabilizers.

The daily dosage of such a preparation for adult human is not particularly limited but is preferably 0.1 to 3,000 mg/kg, more preferably 1 to 300 mg/kg, as the compound of the invention, or the salt or ester thereof, which dosage is to be administered once a day or in a few divided doses a day. The composition can also be used as a pharmaceutical product for domestic and pet animals and the daily dosage for this application is not particularly limited but is preferably 0.1 to 3,000 mg/kg as the compound of the invention, or the salt or ester thereof.

The compound of the present invention, and the salt, ester and composition thereof comprise edible licorice extract components or related compounds thereof, and are considered to be low toxic. The licorice extract obtained by the present invention can also be added to foods to which sweetness or the like is an obstacle. It is highly stable as compared with highly unsaturated fatty acids which are conventionally reported PPARγ ligands, and is superior since it is possible to be made into a form suitable for foods and pharmaceutical compositions.

Thus, a composition which comprises, as an active ingredient, at least one compound selected from among the compound represented by the formula (1), compounds represented by the formula (4) and compounds represented by the formula (5), or a salt or ester thereof is effective for the prevention and/or treatment of metabolic syndrome and for the prevention and/or treatment of insulin resistance syndrome.

Furthermore, a method for the prevention and/or treatment of metabolic syndrome and a method for the prevention and/or treatment of insulin resistance syndrome of the present invention comprise using (administering or applying) at least one compound selected from among the compound represented by the formula (1), compounds represented by the formula (4) and compounds represented by the formula (5), or a salt or ester thereof.

In addition, the use of at least one compound selected from among the compound represented by the formula (1), compounds represented by the formula (4) and compounds represented by the formula (5), or a salt or ester thereof for the preparation of a composition for the prevention and/or treatment of metabolic syndrome and for the preparation of a composition for the prevention and/or treatment of insulin resistance syndrome is within the scope of the present invention.

### EFFECT OF THE INVENTION

According to the present invention, peroxisome proliferator-activated receptor γ (PPARγ) ligand agents and the composition comprising the same are provided easily and efficiently. The ligand agent and the composition of the present invention are effective for the prevention and/or treatment of metabolic syndrome and insulin resistance syndrome.

### BEST MODE FOR CARRYING OUT THE INVENTION

Hereinafter, the present invention is described in more detail by reference to the Examples, but the scope of the present invention is not limited to these Examples.

### Example 1

### Extraction and Isolation of Compounds from Licorice

Four kilograms of licorice (the root and stolons of Glycyrrhiza glabra) were extracted with two 2.5-liter portions of 95% ethanol (45° C., 2 hours per run). The extracts were combined and concentrated under reduced pressure to remove the solvent. An extract weighing 120.8 g was obtained. This extract was subjected to normal phase silica gel column chromatography (hereinafter, CC), followed by elution with chloroform-methanol (19:1, 9:1, 2:1) and methanol to give a total of 64 fractions (500 mL per fraction). Fractions Nos. 1 to 16 were combined and subjected to normal phase silica gel CC, followed by elution with chloroform-methanol (99:1) to give a total of 50 fractions (100 mL per fraction). Fractions Nos. 33 to 37 were subjected to reversed phase silica gel CC, followed by elution with methanol-water (4:1) to give a total of 88 fractions (100 mL per fraction). Fractions Nos. 1 to 10 were subjected to normal phase silica gel CC, followed by elution with chloroform-methanol (100:1, 50:1, 20:1) to give a total of 62 fractions (100 mL per fraction). Fractions Nos. 34 to 42 were subjected to reversed phase silica gel CC, followed by elution with acetonitrile-water (2:3) to give a total of 40 fractions (50 mL per fraction). Fractions Nos. 20 to 22 were purified using a preparatory HPLC equipped with a reversed phase silica gel column (eluent: methanol-water, 7:3) to give a total of 3 fractions. Fraction No. 2 (retention time: 44 minutes) was purified using a preparatory HPLC equipped with a reversed silica gel column (eluent: acetonitrile-water, 1:1) to give Compound 1 (8.0 mg) (retention time: 87 minutes). The fraction number or numbers mentioned above refer to the number or numbers of the fractions obtained just by the respective preceding procedures (hereinafter the same shall apply).

Four kilograms of licorice (the root and stolons of Glycyrrhiza glabra) were extracted with two 2.5-liter portions of 95% ethanol (45° C., 2 hours per run). The extracts were combined and concentrated under reduced pressure to remove the solvent. An extract weighing 120.8 g was obtained. This extract was subjected to normal phase silica gel CC, followed by elution with chloroform-methanol (19:1, 9:1, 2:1) and methanol to give a total of 64 fractions (500 mL per fraction) Fractions Nos. 1 to 16 were combined and subjected to normal phase silica gel CC, followed by elution with chloroform-methanol (99:1) to give a total of 50 fractions (100 mL per fraction). Fractions Nos. 2 to 5 were combined and subjected to normal phase silica gel CC, followed by elution with hexane-acetone (3:1) to give a total of 80 fractions (100 mL per fraction). Fractions Nos. 15 to 17 were subjected to reversed phase silica gel CC, followed by elution with acetonitrile-water (3:1) to give a total of 44 fractions (50 mL per fraction). Fraction Nos. 9 to 13 were purified using a preparatory HPLC equipped with a reversed silica gel column (eluent: acetonitrile-water, 2:1) to give Compound 2 (7.8 mg) (retention time: 87 minutes).

Four kilograms of licorice (the root and stolons of Glycyrrhiza glabra) were extracted with two 2.5-liter portions of 95% ethanol (45° C., 2 hours per run). The extracts were combined and concentrated under reduced pressure to remove the solvent. An extract weighing 120.8 g was obtained. This extract was subjected to normal phase silica gel CC, followed by elution with chloroform-methanol (19:1, 9:1, 2:1) and methanol to give a total of 64 fractions (500 mL per fraction) Fractions Nos. 1 to 16 were combined and subjected to normal phase silica gel CC, followed by elution with chloroform-methanol (99:1) to give a total of 50 fractions (100 mL per fraction). Fractions Nos. 2 to 5 were combined and subjected to normal phase silica gel CC, followed by elution with hexane-acetone (3:1) to give a total of 80 fractions (100 mL per fraction). Fractions Nos. 29 to 38 among the 80 fractions were combined and subjected to reversed phase silica gel CC, followed by elution with acetonitrile-water (3:1) to give a total of 44 fractions (50 mL per fraction). Fraction Nos. 5 to 7 were purified using a preparatory HPLC equipped with a reversed silica gel column (eluent: acetonitrile-water, 2:1) to give Compound 3 (7.3 mg) (retention time: 33 minutes).

As a result of structural analysis, Compound 2 was identified as shinflavanone. In structural identification of that compound, reference was made to the spectral data described in I. Kitagawa et al., Chemical & Pharmaceutical Bulletin, 42, 1056-1062 (1994).

Compound 1 is a novel compound, and detailed structural analysis based on various two-dimensional NMR data revealed that its structure is 3,4,3′,4′-tetrahydroxy-2-methoxy-5′-γ,γ-dimethylallylchalcone.

Description of and spectral data for Compound 1:

brown powders, C₂₁H₂₂O₆

HR-ESI-MS m/z: 371.1487

(calculated value; C₂₁H₂₃O₆: 371.1495)

UV l max (methanol) nm: 368 (4.45), 257 (4.11)

IR (NaCl plate) cm⁻¹: 3375, 2970, 1699, 1642, 1595, 1566, 1507, 1469, 1434, 1298, 1212, 1173, 1053, 987, 941, 854.

¹H-NMR (acetone D) ppm: 7.91 (1H, d, J=15.7 Hz, H-b), 7.67 (1H, d, J=15.7 Hz, H-a), 7.55 (1H, d, J=1.9 Hz, H-6′), 7.51 (1H, d, J=1.9 Hz, H-2′), 7.25 (1H, d, J=8.5 Hz, H-6), 6.72 (1H, d, J=8.5 Hz, H-5), 3.88 (3H, s, 2-OMe), 3.43 (2H, d, J=7.3 Hz, H-1″), 1.77 and 1.75 (each 3H, s, Me-4″ and Me-5″).

¹³C-NMR (acetone D) ppm: 188.0 (C-b′), 149.0 (C-4), 148.7 (C-2), 148.3 (C-4′), 144.6 (C-3′), 138.7 (C-3), 138.4 (C-b), 132.3 (C-3″), 130.7 (C-1′), 128.2 (C-5′), 122.9 (C-2″), 122.8 (C-6′), 121.0 (C-1), 120.5 (C-a), 119.6 (C-6), 113.1 (C-2′), 112.0 (C-5), 61.0 (OMe), 25.4 (C-4″), 17.4 (C-5″).

Compound 3 is a novel compound, and detailed structural analysis based on various two-dimensional NMR data revealed that its structure is 3,2′-dihydroxy-4′-methoxy-6″,6″-dimethylpyrano[2″,3″:7,8] isoflavanone.

Description of and spectral data for Compound 3:

white powders, C₂₁H₂₀O₆

HR-ESI-MS m/z: 369.1320 (calculated value; C₂₁H₂₁O₆: 369.1338 UV l max (methanol) nm: 313 (3.98), 268 (4.56).

IR (NaCl plate) cm⁻¹: 3363, 2976, 2932, 1669, 1620, 1596, 1577, 1510, 1463, 1438, 1372, 1274, 1212, 1165, 1113, 1080, 1023, 961, 887, 839.

¹H-NMR (acetone D) ppm: 7.71 (1H, d, J=8.7 Hz, H-5), 7.39 (1H, d, J=8.6 Hz, H-6′), 6.65 (1H, d, J=10.1 Hz, H-4″), 6.52 (1H, d, J=8.7 Hz, H-6), 6.44 (1H, dd, J=8.6, 2.4 Hz, H-5′), 6.41 (1H, d, J=2.4 Hz, H-3′), 5.76 (1H, d, J=10.1 Hz, H-5″), 4.99 (1H, d, J=11.8 Hz, H-2a), 4.41 (1H, d, J=11.8 Hz, H-2b), 3.74 (3H, s, 4′-OMe), 1.46 and 1.45 (each 3H, s, Me-7 “and Me-8”).

¹³C-NMR (acetone D) ppm: 189.9 (C-4), 161.6 (C-4′), 159.6 (C-7), 157.4 (C-8a), 156.8 (C-2′), 129.6 (C-5), 128.9 (C-5″), 128.3 (C-6′), 117.5 (C-1′), 115.6 (C-4″), 113.9 (C-4-a), 111.5 (C-6), 109.5 (C-8), 105.3 (C-5′), 102.7 (C-3′), 77.9 (C-6″), 74.5 (C-3), 74.4 (C-2), 55.0 (OMe), 27.9×2 (C-7″, C-8″).

The structures of the compounds 1, 2, 3 mentioned above are shown in Table 1.

TABLE 1

| Compound | Structure |
|---|---|
| Compound 13,4,3′,4′-tetrahydroxy-2-methoxy-5′-γ,γ-dimethylallylchalcone | 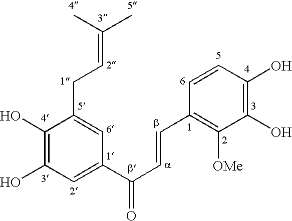 |
| Compound 2shinflavanone | 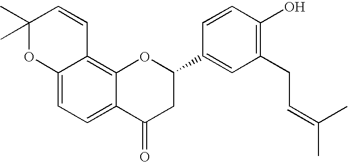 |
| Compound 33,2′-dihydroxy-4′-methoxy-6″,6″-dimeth-ylpyrano[2″,3″:7,8] isoflavanone | 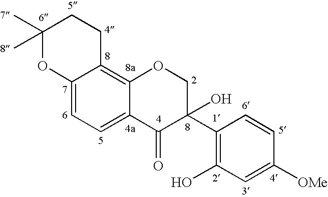 |

### Example 2

### PPARγ Ligand Activity Measurement

CV-1 cells (male African green monkey kidney-derived cultured cells) were distributed into wells of a 96-well culture plate (6×10³ cells/well) and cultured under conditions of 37° C. and 5% CO₂ for 24 hours. The medium used was DMEM (Dulbecco's modified Eagle medium; GIBCO) containing 10% FBS (fetal bovine serum), 10 ml/L of penicillin-streptomycin solution (5,000 IU/ml and 5,000 μg/ml, respectively; GIBCO) and 37 mg/L of ascorbic acid (Wako Pure Chemical Industries). After rinsing with OPTI-MEM (GIBCO), the cells were transfected with pM-mPPARγ and 4×UASg-luc using Lipofectamine Plus (GIBCO). The pM-mPPARγ is a chimera protein expression plasmid resulting from binding of the yeast-derived transcription factor GAL4 gene (amino acid sequence 1 to 147) to the mouse PPARγ ligand binding site gene (amino acid sequence 174 to 475), and the 4×UASg-luc is a reporter plasmid resulting from four repeated insertions, upstream of the luciferase gene, of the response element (UASg) of GAL4. About 24 hours after transfection, the medium was replaced with the one containing a sample (Compound 1, 2 or 3, or positive control troglitazone), followed by 24 hours of cultivation (n=4).

Each sample in the assay group was dissolved in dimethyl sulfoxide (DMSO), and DMSO was used in the no-treatment control group. The solution or DMSO was added to the medium at a level of 1/1000. After washing the cells with Ca- and Mg-containing phosphate buffered saline (PBS⁺), LucLite (Packard) was added to each well, and the luciferase-due luminescence intensity was measured using Packard's TopCount microplate scintillation/luminescence counter.

Using pM (plasmid deprived of the PPARγ ligand binding site gene) in lieu of pM-mPPARγ, measurements in a control group were carried out in the same manner as in the above assay group.

For each sample, the ratio in mean luminescence intensity (n=4) between the assay group and control group (assay group/control group ratio) was calculated, and the specific activity relative to the no-treatment control group was regarded as the PPARγ ligand activity of the sample. When the specific activity was 1.5 or above, the sample was evaluated as having PPARγ ligand activity.

The results of PPARγ ligand activity measurements with Compounds 1 to 3 obtained in Example 1 are shown in Table 2. The result obtained by using troglitazone (Sankyo) as a positive control is also shown in Table 2. PPARγ ligand activity comparison among the compounds tested revealed that Compounds 1, 2 and 3 are stronger in PPARγ ligand activity as compared with troglitazone at 0.5 μM.

TABLE 2

| Compound | Specific activity |
|---|---|
| Troglitazone (0.5 μM) | 1.82 |
| Compound 1 (10 μg/ml) | 3.58 |
| Compound 2 (10 μg/ml) | 3.62 |
| Compound 3 (20 μg/ml) | 1.90 |

### INDUSTRIAL APPLICABILITY

According to the present invention, peroxisome proliferator-activated receptor γ (PPARγ) ligand agents and the composition comprising the same are provided easily and efficiently. The ligand agent and the composition of the present invention are effective for the prevention and/or treatment of metabolic syndrome and insulin resistance syndrome.

1. A compound represented by the formula (1) or a salt or ester thereof.

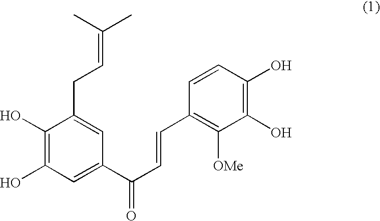

2. A compound represented by the formula (2) or a salt or ester thereof.

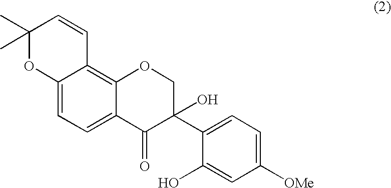

3. A PPARγ ligand agent
which comprises, as an active ingredient, at least one compound selected from among the compound represented by the formula (1), compounds represented by the formula (4) and compounds represented by the formula (5), or a salt or ester thereof: 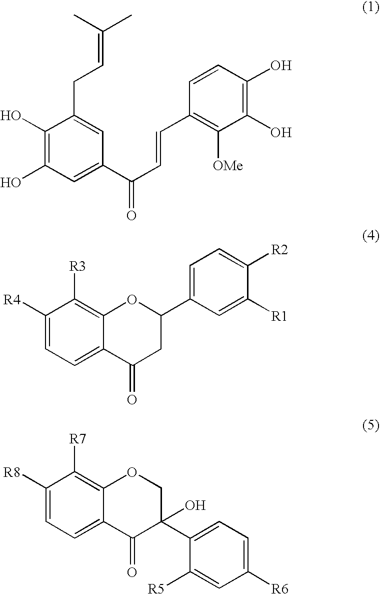
[In the above formulae, at least one of R1 to R4 is a prenyl group or at least one pair of neighboring two groups together represent —CH═CHC(CH₃)₂O— to form a six-membered ring and the others each independently represents H, OH, OCH₃ or CHO. R5 and R6 each independently represents H, OH or OCH₃. At least one of R7 and R8 is a prenyl group or R7 and R8 together represent —CH═CHC(CH₃)₂O— to form a six-membered ring; the other represents H, OH, OCH₃ or CHO.]

4. A composition for the prevention and/or treatment of metabolic syndrome,
which comprises, as an active ingredient, at least one compound selected from among the compound represented by the formula (1), compounds represented by the formula (4) and compounds represented by the formula (5), or a salt or ester thereof. 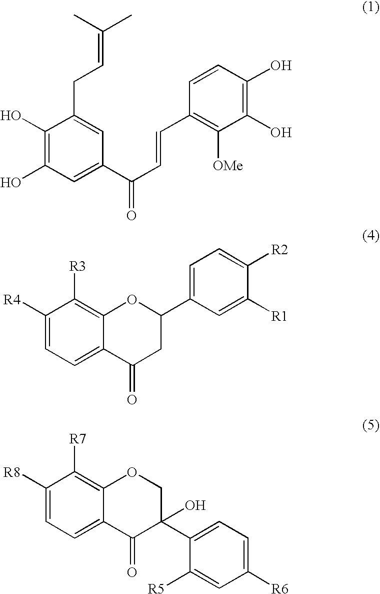
[In the above formulae, at least one of R1 to R4 is a prenyl group or at least one pair of neighboring two groups together represent —CH═CHC(CH₃)₂O— to form a six-membered ring and the others each independently represents H, OH, OCH₃ or CHO. R5 and R6 each independently represents H, OH or OCH₃. At least one of R7 and R8 is a prenyl group or R7 and R8 together represent —CH═CHC(CH₃)₂O— to form a six-membered ring; the other represents H, OH, OCH₃ or CHO.]

5. The composition for the prevention and/or treatment of metabolic syndrome according to claim 4
wherein the compound represented by the formula (4) is a compound represented by the formula (6).

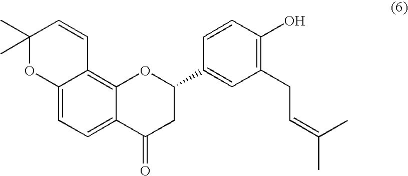

6. The composition for the prevention and/or treatment of metabolic syndrome according to claim 4
wherein the compound represented by the formula (5) is the compound represented by the formula (2).

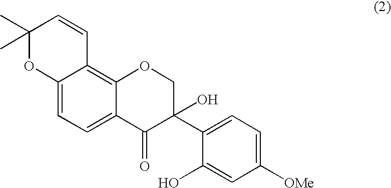

7. The composition for the prevention and/or treatment of metabolic syndrome according to claim 4
which comprises at least one compound selected from among the compound represented by the formula (1), compounds represented by the formula (4) and compounds represented by the formula (5), or a salt or ester thereof, the content thereof being not lower than 0.0000001% by weight but lower than 100% by weight.

8. A composition for the prevention and/or treatment of insulin resistance syndrome,
which comprises, as an active ingredient, at least one compound selected from among the compound represented by the formula (1), compounds represented by the formula (4) and compounds represented by the formula (5), or a salt or ester thereof 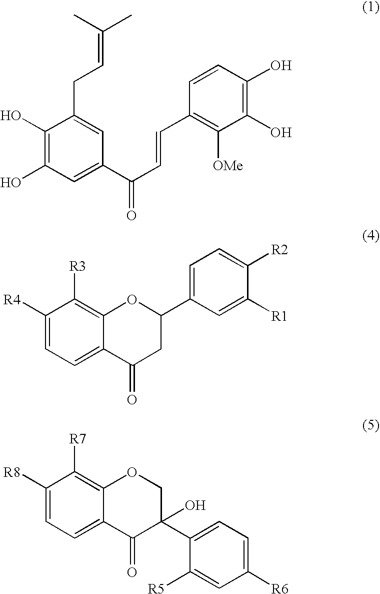
[In the above formulae, at least one of R1 to R4 is a prenyl group or at least one pair of neighboring two groups together represent —CH═CHC(CH₃)₂O— to form a six-membered ring and the others each independently represents H, OH, OCH₃ or CHO. R5 and R6 each independently represents H, OH or OCH₃. At least one of R7 and R8 is a prenyl group or R7 and R8 together represent —CH═CHC(CH₃)₂O— to form a six-membered ring; the other represents H, OH, OCH₃ or CHO.]

9. The composition for the prevention and/or treatment of insulin resistance syndrome according to claim 8
wherein the compound represented by the formula (4) is a compound represented by the formula (6).

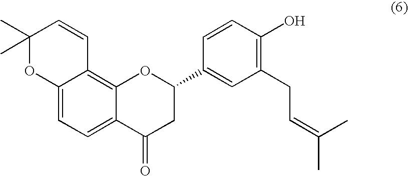

10. The composition for the prevention and/or treatment of insulin resistance syndrome according to claim 8
wherein the compound represented by the formula (5) is the compound represented by the formula (2).

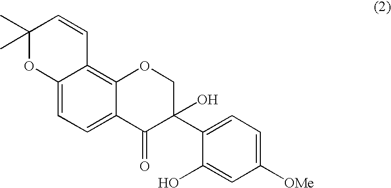

11. The composition for the prevention and/or treatment of insulin resistance syndrome according to claim 8
which comprises at least one compound selected from among the compound represented by the formula (1), compounds represented by the formula (4) and compounds represented by the formula (5), or a salt or ester thereof, the content thereof being not lower than 0.0000001% by weight but lower than 100% by weight.

12. The composition according to 4
which is to be eaten or drunk.

13. The composition according to 4
which is used for medical or medicinal purposes.

14. The composition according to 4
which is applied to domestic animals or pets.

15. A method for the prevention and/or treatment of metabolic syndrome,
which comprises using at least one compound selected from among the compound represented by the formula (1), compounds represented by the formula (4) and compounds represented by the formula (5), or a salt or ester thereof: 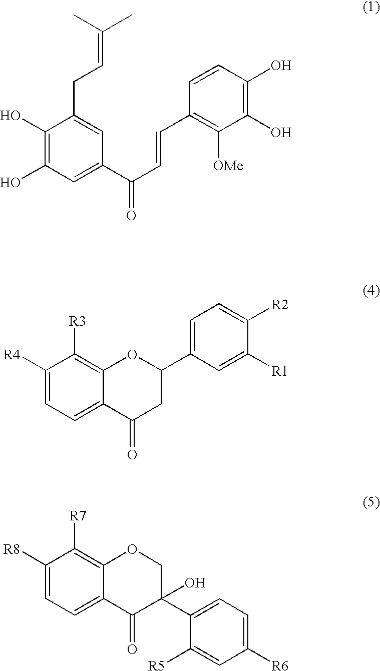
[In the above formulae, at least one of R1 to R4 is a prenyl group or at least one pair of neighboring two groups together represent —CH═CHC(CH₃)₂O— to form a six-membered ring and the others each independently represents H, OH, OCH₃ or CHO. R5 and R6 each independently represents H, OH or OCH₃. At least one of R7 and R8 is a prenyl group or R7 and R8 together represent —CH═CHC(CH₃)₂O— to form a six-membered ring; the other represents H, OH, OCH₃ or CHO.]

16. A use of at least one compound selected from among the compound represented by the formula (1), compounds represented by the formula (4) and compounds represented by the formula (5), or a salt or ester thereof for the preparation of a composition for the prevention and/or treatment of metabolic syndrome: 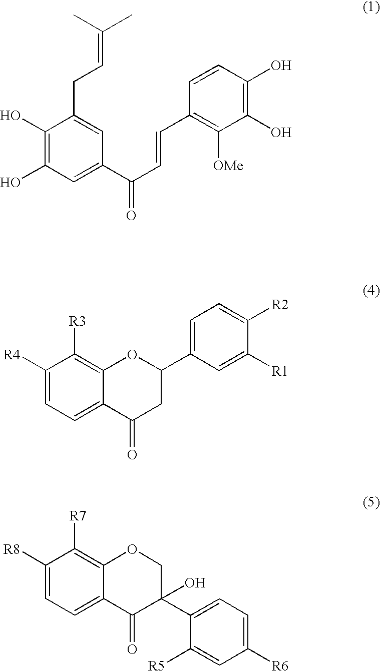
[In the above formulae, at least one of R1 to R4 is a prenyl group or at least one pair of neighboring two groups together represent —CH═CHC(CH₃)₂O— to form a six-membered ring and the others each independently represents H, OH, OCH₃ or CHO. R5 and R6 each independently represents H, OH or OCH₃. At least one of R7 and R8 is a prenyl group or R7 and R8 together represent —CH═CHC(CH₃)₂O— to form a six-membered ring; the other represents H, OH, OCH₃ or CHO.]

17. A method for the prevention and/or treatment of insulin resistance syndrome,
which comprises using at least one compound selected from among the compound represented by the formula (1), compounds represented by the formula (4) and compounds represented by the formula (5), or a salt or ester thereof: 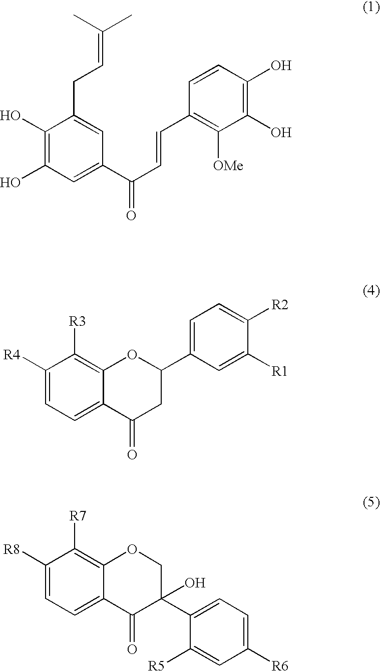
[In the above formulae, at least one of R1 to R4 is a prenyl group or at least one pair of neighboring two groups together represent —CH═CHC(CH₃)₂O— to form a six-membered ring and the others each independently represents H, OH, OCH₃ or CHO. R5 and R6 each independently represents H, OH or OCH₃. At least one of R7 and R8 is a prenyl group or R7 and R8 together represent —CH═CHC(CH₃)₂O— to form a six-membered ring; the other represents H, OH, OCH₃ or CHO.]

18. A use of at least one compound selected from among the compound represented by the formula (1), compounds represented by the formula (4) and compounds represented by the formula (5), or a salt or ester thereof for the preparation of a composition for the prevention and/or treatment of insulin resistance syndrome: 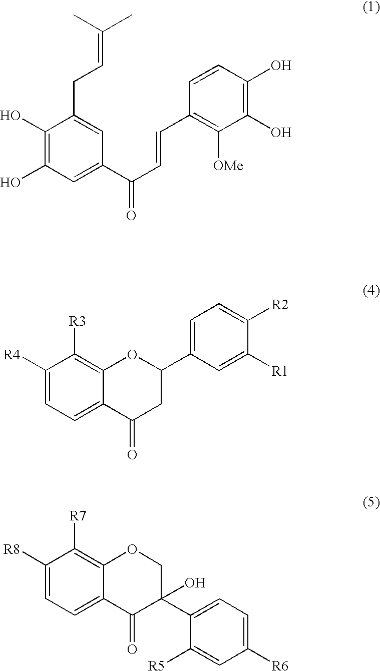
[In the above formulae, at least one of R1 to R4 is a prenyl group or at least one pair of neighboring two groups together represent —CH═CHC(CH₃)₂O— to form a six-membered ring and the others each independently represents H, OH, OCH₃ or CHO. R5 and R6 each independently represents H, OH or OCH₃. At least one of R7 and R8 is a prenyl group or R7 and R8 together represent —CH═CHC(CH₃)₂O— to form a six-membered ring; the other represents H, OH, OCH₃ or CHO.]

19. The composition for the prevention and/or treatment of metabolic syndrome according to claim 5
which comprises at least one compound selected from among the compound represented by the formula (1), compounds represented by the formula (4) and compounds represented by the formula (5), or a salt or ester thereof, the content thereof being not lower than 0.0000001% by weight but lower than 100% by weight.

20. The composition for the prevention and/or treatment of insulin resistance syndrome according to claim 9
which comprises at least one compound selected from among the compound represented by the formula (1), compounds represented by the formula (4) and compounds represented by the formula (5), or a salt or ester thereof, the content thereof being not lower than 0.0000001% by weight but lower than 100% by weight.
